(19) United States
(12) Patent Application Publication
(10) Pub. No.: US 2023/0073576 A1
(43) Pub. Date: Mar. 9, 2023

(54) BLOOD TRANSFUSION TRACKING AND DECISION SUPPORT

(71) Applicants: Jeremy Cannon, Philadelphia, PA (US); Christopher Meador, Missouri City, TX (US)

(72) Inventors: Jeremy Cannon, Philadelphia, PA (US); Christopher Meador, Missouri City, TX (US)

(21) Appl. No.: 17/893,586

(22) Filed: Aug. 23, 2022

Related U.S. Application Data
(60) Provisional application No. 63/241,402, filed on Sep. 7, 2021.

(51) Int. Cl. G16H 40/20; A61M 1/02; G16H 20/40; G16H 10/60; G16H 20/10

(52) U.S. Cl. CPC G16H 40/20; A61M 1/02; G16H 10/60; G16H 20/10; G16H 20/40

(57) ABSTRACT

A method for tracking and supporting a blood transfusion to a patient. The method includes receiving, by a computing device, current blood product data that identifies a blood product being administered to the patient; updating a current ratio of blood products administered to the patient based on the current blood product data; comparing the current ratio of blood products administered to the patient to a target ratio of blood products to be provided to the patient; determining a next blood product to administer to the patient based on the comparison of the target ratio to the current ratio; and providing, by the computing device, an indication of the determined next blood product.

### CROSS REFERENCE TO RELATED APPLICATIONS

The present application claims priority to U.S. Provisional Pat. Application No. 63/241,402, filed Sep. 7, 2021, entitled “Blood Transfusion Tracking and Decision Support,” which is hereby incorporated herein by reference in its entirety.

### STATEMENT REGARDING FEDERALLY-SPONSORED RESEARCH

This invention was made with government support under W81XWH-18-C-0163 awarded by the United States Army. The government has certain rights in the invention.

### BACKGROUND

Blood transfusion tracking, particularly when patients are in critical status and need blood products rapidly (e.g., hemorrhagic shock with significant ongoing blood loss), has proven challenging. In these emergency situations, care providers often order a massive transfusion protocol (MTP), which initiates several actions, including: the administration of whole blood, packed red blood cells, plasma, and/or platelets (as used herein, “blood products” may refer to any or all of these) in a balanced fashion to the patient; and a standing order with a blood bank to prepare and send a container(s) of pre-determined quantities of blood products to the patient’s location, and to do so continually until the massive transfusion order is cancelled.

### SUMMARY

In accordance with at least one example of the disclosure, a method is provided for tracking and supporting a blood product transfusion to a patient. The method includes receiving, by a computing device, current blood product data that identifies a blood product being administered to the patient; updating a current ratio of blood products administered to the patient based on the current blood product data; comparing the current ratio of blood products administered to the patient to a target ratio of blood products to be provided to the patient; determining a next blood product to administer to the patient based on the comparison of the target ratio to the current ratio; and providing, by the computing device, an indication of the determined next blood product.

In another example of the disclosure, a non-transitory, computer-readable medium that contains instructions that, when executed by a processor of a computing device, cause the computing device to be configured to receive current blood product data that identifies a blood product being administered to a patient, update a current ratio of blood products administered to the patient based on the current blood product data, compare the current ratio of blood products administered to the patient to a target ratio of blood products to be provided to the patient, determine a next blood product to administer to the patient based on the comparison of the target ratio to the current ratio, and provide an indication of the determined next blood product.

In yet another example of the description, a device that includes a processor, a display unit coupled to the processor, and a memory coupled to the processor. The memory is configured to store instructions that, when executed by the processor, cause the device to be configured to receive current blood product data that identifies a blood product being administered to a patient, update a current ratio of blood products administered to the patient based on the current blood product data, compare the current ratio of blood products administered to the patient to a target ratio of blood products to be provided to the patient, determine a next blood product to administer to the patient based on the comparison of the target ratio to the current ratio, and provide an indication of the determined next blood product.

### BRIEF DESCRIPTION OF THE DRAWINGS

For a detailed description of various examples, reference will now be made to the accompanying figures, in which:

FIG. 1 is a schematic block diagram of a computing device including blood transfusion tracking and decision support software in accordance with various examples;

FIG. 2 is a schematic block diagram of a system for blood transfusion tracking and decision support in accordance with various examples;

FIG. 3 is a flow chart of a method for providing blood transfusion tracking and decision support in accordance with various examples; and

FIG. 4 is an example screenshot of a computing device that is configured to provide blood transfusion tracking and decision support in accordance with various examples.

### DETAILED DESCRIPTION

As used herein, “blood products” refers to any or all of whole blood, packed red blood cells, plasma, and platelets. In some cases, a balance (e.g., ratio) of blood products provided to a patient may affect patient survivability. For example, a 1:1:1 ratio of plasma to platelets to red blood cells has been shown to reduce 24-hour mortality by exsanguination relative to other ratios, such as a 1:1:2 ratio. Some multi-center data indicates that, even though providers know that a 1:1:1 ratio improves patient outcomes, in practice, half of the patients in “ultra-massive” resuscitations (e.g., those involving 20 or more units of packed red blood cells) had an imbalanced resuscitation. For example, either the ratio of red blood cells to plasma, or the ratio of red blood cells to platelets were >= 1.5.

Patients with unbalanced resuscitation, in which the ratio of blood products differs from a target ratio by more than a predetermined amount, had correspondingly increased mortality odds ratio. In some cases, a percent of time the resuscitation is unbalanced during the first six hours may also be associated with higher mortality odds ratio, such as when the ratio of red blood cells to plasma is >1.33 for 60% or more of the first six hours. In many cases, it is difficult to achieve a balanced resuscitation, in which blood products are provided to the patient at or near a predetermined ratio (e.g., 1:1:1 as described above).

Blood products are regulated by the Food and Drug Administration (FDA). One FDA regulation for blood products is the need to be labeled according to the Information Standard for Blood and Transport (ISBT) 128 standard. For many blood products prepared for clinical use, the blood bag label has a barcode for each of the following datum: Donor Identification Number (DIN), blood product type, blood type (or “ABORh” value), and expiration date. There are also regulations for storage conditions, such as temperature, and expiration dates.

The practice of administering blood products in acute care hospitals is regulated by the Joint Commission (JC). Patients may be harmed if given the incorrect blood ABORh type. To avoid this harm, two clinicians, including registered nurses, nurse anesthetists, or physicians, should verify that the blood product either matches the patient’s blood type (if determined by laboratory testing), or that the blood product is type O, which is considered a “universal blood type”, because almost all patients may receive type O without a negative reaction. The dual verification (e.g., by two clinicians) also applies to patient identification, to better ensure that the blood products are delivered to the appropriate patient. In some cases, the dual verification by nurses is documented on paper.

Additionally complicating matters, platelets processed using apheresis are taken from a single donor and concentrated to create the equivalent of five or six doses of platelets taken from donated whole blood. These multiple apheresis platelet units are stored within a single bag, whereas individual donor plasma, red blood cells, and whole blood units are stored within a single bag. Pediatric care is further complicated as blood product dosing is typically measured by volume in milliliters based on the Broselow™ Tape weight category. For example, a pediatric patient having a first weight may receive blood products at different doses than another pediatric patient having a second weight. The volumes of each blood component naturally occurring in healthy humans are not equal. For example, a recorded volume of 150 mL red blood cells to 120 mL plasma is closer to a balanced transfusion than 120 mL red blood cells to 120 mL plasma. Accordingly, a pediatric clinician attempting to give a balanced transfusion while tracking blood given by volumes (e.g., according to nursing practice) has a more difficult task than simply counting blood bags.

Deciding whether to initiate a massive transfusion protocol may also be challenging. Often, surgeons or trauma teams make the decision to initiate a massive transfusion based on initial data, experience, and gestalt. However, initiating a massive transfusion when it may not be necessary may tie up the blood bank and may result in blood product spoilage. Some surgeons or trauma teams rely on a variety of massive transfusion “scores” to determine whether to initiate a massive transfusion. For example, the massive transfusion scores estimate the likelihood of a patient needing a qualifying amount of blood products in a certain timeframe, such as 10 units of red blood cells within 24 hours, or three units of red blood cells within a 60-minute period. The Assessment of Blood Consumption (“ABC”) score is one example of a score that does not require laboratory values.

During massive transfusions, certified blood bank technicians must prepare blood products for transfusion. Plasma is commonly stored as “fresh frozen plasma” (“FFP”) to achieve an FDA approved shelf life of approximately one year. The blood bank technician must warm FFP according to FDA regulations in order to prepare it for transfusion. This process may take on the order of 20 minutes, and may be limited to just one bag of FFP per warming equipment (often a fluid bath). Once the FFP is thawed, it is considered “thawed plasma”, it receives a new product code and a much shorter shelf life (expiration date), on the order of five (5) days. If the thawed plasma is not given during the massive transfusion, and that unit of plasma is not given to another patient before expiration, then that product must be disposed per FDA regulations, which also contributes to spoilage. It is important for the blood bank technician to be notified as soon as possible after the physician cancels the massive transfusion order, so that the technician can stop thawing plasma and avoid unnecessary waste.

During massive transfusions, multiple (e.g., four to thirteen) blood products are stored in a cooler, which keeps the blood products as a low, but not frozen, temperature. Clinicians may not need to give all the blood products in a particular cooler. If the blood products (e.g., plasma and red blood cells) are kept continually cool enough, then those products can be returned to the blood bank for use with other patients. If the products get too warm, then the blood must be discarded per FDA regulations, which further contributes to spoilage. After a physician cancels the massive transfusion order, then a member of the clinical team (e.g., a nurse), may notify the blood bank that the massive transfusion order has been cancelled. However, with many tasks to perform in a chaotic situation, the blood bank is often not notified for long periods of time. Certain research has indicated that a primary source of blood product waste during massive transfusions was the failure to timely notify the blood bank of MTP deactivation (e.g., within 60 minutes).

In addition to recording the raw product types (e.g., red blood cells, plasma bags) counted above, the JC best practice is to record: 1) the time at which the blood product administration began, and 2) the time at which the product administration ended. Each blood bag could potentially be identified by a unique combination of DIN and product type. However, due to the chaotic nature of the clinical situation, tracking of individual blood bags may be limited to the timeframe during which the patient was in a particular location. Sometimes, documentation of transfusions is done after the patient has left the location and clinicians may resort to guessing the order in which blood products were transfused to the patient based on which bags were thrown on top of which other bags.

Coagulopathy data may include data generated by viscoelastic hemostatic assays (VHAs) or other medical devices (e.g., TEG® 6 s Hemostasis analyzer (Haemonetics Corp, Boston, MA) or ROTEM® device (Werfen, Bedford, MA)). Such coagulopathy data may be used to fine-tune the resuscitation by indicating when providers should consider giving certain hemostatic adjuncts or adjunct therapeutic products (e.g., fibrinogen concentrate, tranexamic acid (TXA)), or additional units of certain types of blood products (e.g., platelets, cryoprecipitate). However, it may be challenging for care providers to receive the coagulopathy data timely, and also memorize protocols for which adjuncts or protocols to give based on the quantitative values of certain viscoelastic data.

The EMR system also plays an important role in health care by providing the legal-medical record of diagnostic, therapeutic, and procedural data for the patient. JC regulations for health care organizations and health care providers require, among other things, the recording of blood product transfusions, including the blood DIN, the blood product, the time at which the blood transfusion started, the volume of the blood in each blood bag (which may be estimated in practice), and the time at which the blood transfusion is stopped.

Blood product transfusions are supposed to be documented in the EMR. However, idiosyncrasies in EMR system design create barriers to successful recordation and/or documentation of blood product transfusions. For example, in routine, non-emergency situations, blood products may be ordered by physicians individually within the EMR. The blood bank has blood type cross and match data for the patient, and assigns a particular blood product that matches the patient’s blood type to that particular patient. A bedside nurse (or other clinician) scans that blood product, such as with a phone app or a barcode scanner, and the EMR establishes a positive match between the order and the product scanned by the bedside clinician. However, in emergency situations, the blood bank often does not have cross and match data and so will release “uncrossmatched” blood. There may be a delay for blood bank personnel to document the assignment of particular blood products to the patient in the EMR. For example, an EMR may not allow clinicians to timely use a barcode scanner or phone app to scan the blood bag label data for positive matching with uncrossmatched blood, or during massive transfusions. As a work-around, bedside clinicians may need to manually type the DIN (which consists of 13 characters) into the EMR, select the product type, enter the volume, and also enter the start and stop times of transfusion.

Blood bank Laboratory Information Systems (LIS) are used to keep track of blood product inventory and the location, status, and expiration dates of blood products. Some EMR systems may include LIS functionality, while others do not. If the EMR and LIS are separate software systems, then they may not communicate with each other. If the LIS communicates issued blood products to the EMR, the EMR might not communicate back to the LIS when, or whether, the issued blood products were transfused. For these reasons, blood bank technicians may rely on the EMR or on transfusion reports with handwritten data to reconcile blood product status and inventory in the LIS after a massive transfusion event. Blood products need to have a clinical record of being transfused before they can be billed to the patient.

Before and during hemorrhagic resuscitation, providers also need to consider vital signs (e.g., heart rate, blood pressure, SpO2), blood chemistries, and laboratory data (e.g., sodium, chloride, potassium, bicarbonate, blood urea nitrogen, blood pH, lactate, glucose, serum creatinine, hemoglobin, hematocrit, PaO2, base excess; sometimes, co-oximetry and magnesium). This data typically comes from disparate places, which is a shortcoming of many EMR systems. For example, clinical care decisions may be delayed because providers and clinicians access this information in multiple places, which could negatively impact clinical/patient outcomes.

Current practices have several limitations. First, the emergency nature of massive transfusions results in poor teal-time record keeping of blood products administered to a patient. Blood products provided to the patient can be tallied on a piece of paper, on nurse scrubs, or a white board by category or blood product type (e.g., plasma, platelets, red blood cells, cryoprecipitate). However, after many blood products and/or adjunct therapeutic products (i.e., products other than blood products) have been given in quick succession, there can be mismatches between the number of marks on the white board, the number of blood product bags in the area, the number of blood products sent by the blood bank, the number of dual verification paper record sheets, and the number of blood bags recorded in the electronic medical record (EMR). Used blood bags and/or dual verification papers can be misplaced (e.g., fall behind a computer or under other objects, or disposed before counting), someone might not be available to document transfusion data into the EMR simultaneously with the transfusions, and the operating room may need to be quickly turned around for another trauma case.

The process of manually entering DINs is prone to data entry errors, which is problematic for the patient’s legal-medical record. If blood bags or paperwork are lost during emergency care, or a clinician believes that they have already documented that product, but in reality, did not document that product, then it frustrates the blood bank’s ability to reconcile which blood products released to the patient were actually transfused. Blood banks may use the term “presumed transfused” in their inventory management systems when they have issued a product, and the product was not returned to the blood bank, but there is no clinical record of actual transfusion. Also, a best practice for nursing care involves documenting the volume of fluids infused.

In various examples, a system that implements the functionality described herein may include a server and a computing device (e.g., a tablet, smartphone, dedicated portable computing device, and the like) that is communicatively coupled to the server. The computing device is referred to herein as a tablet for simplicity. The server may be located physically remote from a patient care area, while the tablet may be located physically proximate to the patient care area, including being proximate to the patient during a transfusion procedure.

The tablet is configured to receive user inputs; provide outputs such as audible indications, visual indications, alerts, and the like; and process various data to perform the functionality described herein. The server is configured to receive data from the tablet (e.g., user input data) and to provide data to the tablet (e.g., laboratory data). The server is also configured to provide various data processing functionality, and thus to implement the functionality described herein. In some cases, the server also implements an EMR system, while in other cases the server is configured to couple to a server or other computing device that implements the EMR system.

Examples of this description address the foregoing by providing systems (e.g., including server(s), tablet(s), or combinations thereof) and methods for tracking and otherwise supporting a blood transfusion to a patient. In some examples, this includes providing recommendations for, or indications of, blood products to be administered to the patient, such as based on a target ratio of blood products for the particular patient, and a current ratio of blood products already administered to the patient. For example, the target ratio may be 1:1:1 as described above. However, in other examples, the target ratio may be different than 1:1:1 and may be provided by a clinician as an input (e.g., a setting parameter for the transfusion), such as to a graphical user interface (GUI) of the tablet, or based on a physiological parameter of the patient (e.g., weight, height, age, and the like).

The target ratio of blood products for the patient is thus received or otherwise determined, such as by the tablet or a GUI thereof. Current blood product data, or data that identifies a blood product being administered to the patient is also received. For example, a barcode of a bag containing the blood product may be scanned (e.g., by the tablet or by a scanning device coupled thereto), which provides data that identifies the blood product to the tablet. In another example, a radio-frequency identification (RFID) tag associated with the bag containing the blood product may be scanned (e.g., by the tablet or by a RFID scanner coupled thereto), which provides data that identifies the blood product to the tablet. In this description, a blood product may be considered as being administered to the patient once it has been scanned, even though the blood product is not yet physically administered to the patient.

A current ratio of blood products administered to the patient is updated to include the blood product being administered (e.g., the blood product scanned and about to be physically administered to the patient). Before any blood products have been administered to the patient, the current ratio may simply be updated (e.g., from 0 or a default value) to include the current blood product data. The updated current ratio may be stored in a memory of the tablet, provided to the server for storage thereon, or both. An indication of the current ratio may be provided to the user (e.g., on the tablet GUI). The current ratio is compared to the target ratio, and an indication is provided (e.g., on the tablet GUI) for a next blood product to be administered to the patient based on the comparison of the current ratio to the target ratio. For example, if the current ratio indicates that additional plasma should be administered to bring the balance of blood products closer to the target ratio, the indication is for plasma. Similarly, if the current ratio indicates that additional platelets should be administered to bring the balance of blood products closer to the target ratio, the indication is for platelets. In some examples, if the current blood product data identifies an unexpected blood product (e.g., a prior recommendation was for a first blood product, and the blood product being administered, or that has just been scanned, is a second blood product), an alert may be provided (e.g., on the tablet GUI) to indicate to the user that the blood product is not the appropriate one to be administered at this point in the patient’s treatment.

The current blood product data (e.g., identifying the blood product being administered to the patient) may be received from a barcode associated with the blood product being administered (e.g., by the tablet or a barcode scanner coupled thereto), a radio-frequency identification (RFID) tag associated with the blood product being administered (e.g., by the tablet or an RFID scanner coupled thereto), and image data associated with the blood product being administered (e.g., by a camera of the tablet or a camera coupled thereto).

The various recommendations and/or indications discussed above may be provided as audible indications (e.g., using a speaker of the tablet), visual indications (e.g., using a GUI of the tablet), both audible and visual indications, or other types of indications to the user of the tablet, such as a clinical provider.

In some examples, a control signal may also be provided responsive to determining the next blood product, or responsive to a cancellation (e.g., a cancellation order) of a protocol associated with the blood transfusion to the patient, such as the MTP that was ordered for the patient. For example, the tablet may provide a control signal to cause an infusion device (e.g., an infusion pump) to provide the determined next blood product to the patient in an automated fashion. In this example, the control signal may indicate a type of the determined next blood product, a volume of the determined next blood product, a rate of delivery of the next product, or other values usable by the infusion device to automatically administer the next blood product to the patient.

In another example, the tablet may provide a control signal to a blood bank to cause the blood bank to prepare additional blood product(s) that may be administered to the patient, such as after the determined next blood product. For example, the tablet may determine that, based on the determined next blood product, additional blood product(s) will be needed from the blood bank, and thus generates the control signal to preemptively, or more proactively, request those additional blood product(s). In some cases, the control signal is also responsive to an on-hand inventory. For example, if the next blood product is readily available from an on-hand inventory, the blood bank need not begin preparing additional blood product(s). However, if the next blood product is such that the on-hand inventory will fall below a first threshold, then the blood bank should begin preparing additional blood product(s) and the control signal is thus provided. The control signal may be configured to cause equipment at the blood bank to begin warming the additional blood product(s) or otherwise preparing the blood product(s) for transport and administration to the patient. In another example, if the next blood product is such that the on-hand inventory will fall below a second threshold (e.g., lower than the first threshold), then a control signal is provided to cause an order to be placed with a supplier of the blood bank to provide an additional blood product.

In yet another example, the tablet may provide a control signal to the blood bank to cause the blood bank to stop preparing additional blood products according to the MTP order for the patient, such as responsive to a cancellation of the MTP that is indicated (e.g., by a user input) to the tablet. In some cases, the cancellation of an MTP may be relayed to the blood bank in a delayed or otherwise ineffective manner, which can result in the blood bank preparing blood products for a now-cancelled order, thus leading to spoilage of those blood products. Accordingly, in examples of this description, the tablet may provide the control signal to effect the cancellation of the MTP at the blood bank, which may reduce or eliminate blood product spoilage by more effectively and directly communicating the cancellation of the MTP and causing blood bank personnel and/or equipment to modify their behavior accordingly.

In some cases, in what can be a chaotic clinical environment, a blood product administered to the patient may be “double counted”, or otherwise erroneously tallied, which results in an incorrect assessment of the current ratio of blood products that have been administered to the patient, which in turn may result in worse clinical outcomes. Accordingly, in some examples, the tablet is also configured to detect duplicate scans of blood product data. For example, sometimes a barcode, RFID tag, or other data that identifies the blood product being administered may also be mistakenly scanned or received by the tablet more than one time. In the event that a duplicate scan is detected, the blood product should not be counted again (e.g., to determine or update the current ratio of blood products administered to the patient). Accordingly, the ratio of blood products administered to the patient is updated responsive to the current blood product data when it is not duplicate data, and the ratio of blood products administered to the patient is not updated responsive to the current blood product data when it is duplicate data. This reduces the possibility of erroneous calculations of blood products administered to the patient in what can be a chaotic clinical environment. These and other examples are described more fully below, with reference made to the accompanying figures.

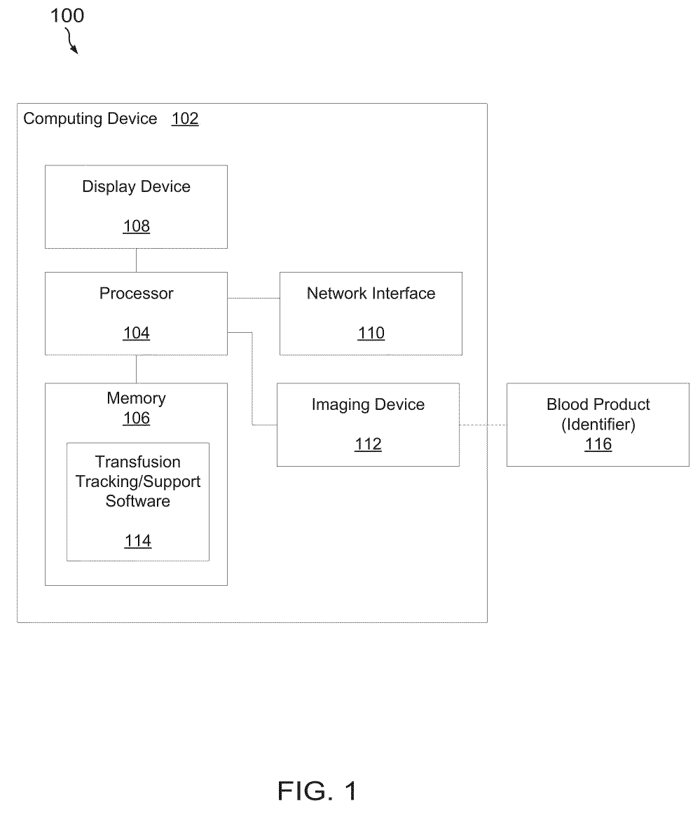

FIG. 1 is a schematic block diagram of a system 100 including a computing device 102 in accordance with various examples. As described above, the computing device 102 may be a tablet, a smartphone, a personal computer such as a laptop, a dedicated portable computing device, and the like. The computing device 102 may also be referred to as a tablet 102 for simplicity, but without sacrificing the generality thereof. The computing device 102 includes a processor 104, which may be a central processing unit (CPU), an application specific integrated circuit (ASIC), or other types of hardware processors that are configured to execute software instructions to perform the functionality described herein. The computing device 102 also includes a memory 106 that is coupled to the processor 104. The memory 106 is configured to store instructions (e.g., transfusion tracking/support software 114) that, when executed by the processor 104, cause the computing device 102 to perform various functionality described herein. The memory 106 may also be configured to store data generated by executing the transfusion tracking/support software 114 for subsequent access and/or retrieval by a user of the computing device 102, or by another computing device that is configured to couple to the computing device 102.

The computing device 102 also includes a display device 108 that is coupled to the processor 104. The display device 108 may be a touch display, in which case the display device 108 may also function as a user input device. The display device 108 may also be a non-touch display, in which case the computing device 102 includes one or more user input devices (not shown for simplicity) that allow a user to interact with the computing device 102 and to implement functionality provided by the processor 104 (e.g., by executing transfusion tracking/support software 114 stored in the memory 106).

The computing device 102 includes a network interface 110, which may facilitate wired and/or wireless connections to one or more networks. The network interface 110 is coupled to the processor 104. The network interface 110 thus enables the computing device 102 to communicate with one or more remote computing devices, such as remote server(s) or other similarly-configured computing devices (e.g., other similar tablets in a clinical environment). The network interface 110 may also facilitate data (e.g., generated by the processor 104 executing transfusion tracking/support software 114) being uploaded for storage, such as to a cloud-based storage solution or to the remote server described previously. The network interface 110 generally facilitates connectivity to myriad other computing devices, storage devices, processing devices, and the like.

The computing device 102 also includes an imaging device 112 in the example of FIG. 1. The imaging device 112 is also coupled to the processor 104, and may be a camera, a barcode scanner, another type of imaging device, or combinations thereof. The imaging device 112 is useful to scan a blood product, a patient identifier (e.g., a medical record number (MRN)), or other identifiers in the clinical setting. In other examples, the computing device 102 may include a radio frequency (RF) transceiver instead of, or in addition to the imaging device 112, such as to acquire data from an RFID tag. By including the imaging device 112 and/or an RF transceiver, the computing device 102 is configured to receive data indicative of a blood product, a patient identifier, other identifiers in the clinical setting (e.g., a user identifier of the computing device 102), and the like. As described further below, these identifying data are usable by the transfusion tracking/support software 114 to facilitate the functionality described herein.

As described, the processor 104 is configured to execute the transfusion tracking/support software 114 stored in the memory 106. In an example, the transfusion tracking/support software 114 comprises instructions stored contained in the memory 106, which is an example non-transitory, computer-readable medium. Accordingly, the transfusion tracking/support software 114, when executed by the processor 104, enables the computing device 102 or tablet 102 to be configured to track and otherwise support a blood transfusion to a patient, such as in response to a MTP order, or another transfusion protocol order.

In some examples, this includes the tablet 102 providing recommendations for, or an indication of blood products to be administered to the patient, such as based on a target ratio of blood products for the particular patient, and a current ratio of blood products already administered to the patient. The indication may be provided on the display device 108 of the tablet 102. As described above, the target ratio can be 1:1:1, or can be different than 1:1:1 and provided or otherwise specified by a clinician as an input to the tablet 102 (e.g., a setting parameter for the transfusion), such as to a graphical user interface (GUI) of the tablet (e.g., a user input to the touch screen display device 108, or other user input device), or based on a physiological parameter of the patient (e.g., weight, height, age, calcium levels, coagulopathy data, and the like). For example, the memory 106 may store a lookup table that associates various physiological parameters with corresponding target ratios, and the target ratio is determined responsive to a user input that indicates certain physiological parameter(s) of the patient.

The target ratio of blood products for the patient is thus received or otherwise determined, such as by the tablet 102 or a GUI thereof. The transfusion tracking/support software 114, when executed, also causes the tablet 102 to receive current blood product data, or data that identifies a blood product being administered to the patient. In FIG. 1, the blood product is represented as 116, which may be a bag containing the blood product and having a barcode that can be scanned by the imaging device 112, which provides data that identifies the blood product to the tablet 102. In another embodiment, the blood product 116 includes an RFID tag that may be scanned by an RF transceiver of the tablet 102. Irrespective of the particular form of identifier of the blood product 116, as described above, a blood product may be considered as being administered to the patient once it has been scanned by the tablet 102, even though the blood product is not yet physically administered to the patient.

The transfusion tracking/support software 114, when executed, also causes the tablet 102 to update a current ratio of blood products administered to the patient to include the blood product being administered (e.g., the blood product scanned and about to be physically administered to the patient). As described, the current ratio may simply be initialized to 0 or a default value before any blood products have been administered to the patient. The updated current ratio can be stored in the memory 106, provided by way of the network interface 110 to a server for storage thereon, or both. In some examples, the display device 108 is configured to provide the indication of the current ratio to the user (e.g., on the tablet 102 GUI).

The transfusion tracking/support software 114, when executed, also causes the tablet 102 to compare the current ratio to the target ratio, and provide an indication (e.g., on the tablet 102 GUI on the display device 108) for a next blood product to be administered to the patient based on the comparison of the current ratio to the target ratio. For example, if the current ratio indicates that additional plasma should be administered to bring the balance of blood products closer to the target ratio, the indication on the display device 108 is for plasma (which may also include a volume thereof, such as a dose in mL that is based on the weight of the patient, which may be useful for pediatric patients in some examples). Similarly, if the current ratio indicates that additional platelets should be administered to bring the balance of blood products closer to the target ratio, the indication on the display device 108 is for platelets (which may also include a volume thereof). In some examples, if the scanned current blood product data identifies an unexpected blood product (e.g., the tablet 102 provided a prior recommendation for a first blood product, and the blood product being administered, or that has just been scanned, is a second blood product), the tablet 102 may be configured to provide an alert (e.g., on the tablet GUI on the display device 108, or through a speaker of the tablet 102) to indicate to the user that the blood product is not the appropriate one to be administered at this point in the patient’s treatment.

As described, current blood product data can be received by the tablet 102 by scanning a barcode associated with the blood product 116 being administered using the imaging device 112 (e.g., by the tablet 102 or a barcode scanner coupled thereto), a radio-frequency identification (RFID) tag associated with the blood product 116 being administered (e.g., by the tablet 102 or an RFID scanner/RF transceiver coupled thereto), and image data associated with the blood product 116 being administered (e.g., by the imaging device 112 of the tablet 102 or a camera coupled thereto).

The various recommendations and/or indications discussed above can be provided as audible indications (e.g., using a speaker of the tablet 102), visual indications (e.g., using a GUI on the display device 108 of the tablet 102), both audible and visual indications, or other types of indications to the user of the tablet 102, such as a clinical provider.

The transfusion tracking/support software 114, when executed, may also cause the tablet 102 to detect duplicate scans of blood product data. For example, sometimes a barcode, RFID tag, or other data that identifies the blood product 116 being administered may also be mistakenly scanned or received by the tablet 102 more than one time. In the event that a duplicate scan is detected, the blood product 116 should not be counted again (e.g., to determine or update the current ratio of blood products administered to the patient). The processor 104 is configured to update the ratio of blood products administered to the patient responsive to the current blood product data when it is not duplicate data (i.e., when the scanned blood product identifier 116 has not been scanned previously). The processor 104 is also configured not to update the ratio of blood products administered to the patient responsive to the current blood product data when it is duplicate data (i.e., when the scanned blood product identifier 116 has been scanned previously). Accordingly, the transfusion tracking/support software 114 enables the tablet 102 to reduce the possibility of erroneous calculations of blood products administered to the patient in what can be a chaotic clinical environment.

In some examples, the transfusion tracking/support software 114, when executed, may also cause the tablet 102 to receive patient identification information, such as for proper transfusion documentation or to permit the tablet 102 to be used in conjunction with administering blood products to more than one patient in a particular clinical setting. For example, the patient identification information can be received via one or more barcodes associated with the patient (e.g., by the imaging device 112 or a barcode scanner coupled to the tablet 102), an RFID tag associated with the patient (e.g., by the tablet 102 or an RFID scanner/RF transceiver coupled thereto), image data associated with the patient (e.g., by the imaging device 112 of the tablet 102 or a camera coupled thereto), or other means of data acquisition. The patient identification information may include the patient’s name, date of birth, certain vital signals or information, medical record number, encounter number, other identifying number(s), and the like.

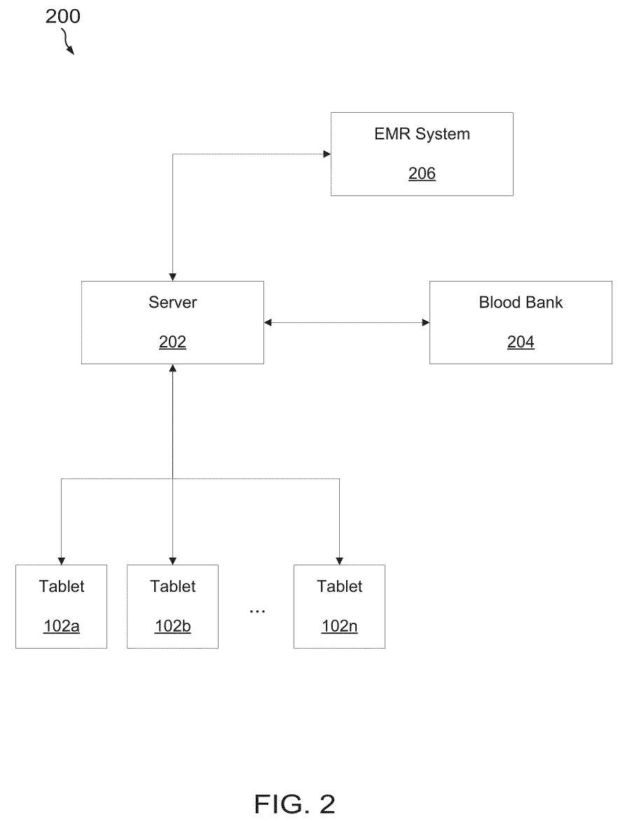

In some cases, the processor 104 executing the transfusion tracking/support software 114 may cause the tablet 102 to provide an indication of a total number and/or volume of blood products administered to the patient responsive to receiving the patient identification data, which enables a clinical provider to quickly ascertain a status of the patient’s blood transfusion. In some examples, the total number and/or volume of blood products administered to the patient includes those blood products scanned by other tablets (but for the same patient) in different locations of the hospitals. For example, the tablet 102 may be configured to provide data to a server (such as the server 202 described below with respect to FIG. 2) that enables the server to aggregate a master data record for a particular patient (e.g., associated with the patient identification data). Previously, other tablets in different locations may have contributed to the master data record for the patient, and thus this data is accessible by the tablet 102 when receiving the patient identification data. Also, subsequently, other tablets in different locations may access the master data record for the patient, which will be updated to include transfusion activities tracked by the particular tablet 102. Accordingly, the transfusion tracking/support software 114 enables the tablet 102 to track whether a patient has received a balanced transfusion across multiple locations where a massive transfusion may occur (e.g., emergency department/trauma bay, operating room, and intensive care unit (ICU)). The transfusion tracking/support software 114 may include a location setting that allows transfusion data to be associated with the location in which the blood product(s) are provided. Further, if a clinician in a particular location routinely administers unbalanced transfusions, the multi-location data collected by the transfusion tracking/support software 114 enables efficient identification of such a clinician for further training.

In some examples, executing the transfusion tracking/support software 114 causes the tablet 102 to provide the count, and/or volume, of blood products and/or hemostatic adjuncts given in the current location, or in all locations for that patient identifier or identifiers. In some examples the tablet 102 provides the ratio of blood products given in the current location, or in all locations for that patient identifier or identifiers.

If the tablet 102 receives patient identification information for a first patient, then the above-described functionality is with respect to that first patient. For example, the target ratio is for the first patient, the current ratio of blood products is for the first patient, and the indication of the next blood product is for the first patient. Conversely, if the tablet 102 receives patient identification information for a second patient, then the above-described functionality is with respect to that second patient. For example, the target ratio is for the second patient, the current ratio of blood products is for the second patient, and the identification of the next blood product is for the second patient. Accordingly, either or both of the tablet 102 and the server coupled thereto are configured to store data for multiple patients and to provide blood transfusion recommendations for multiple patients.

In some examples, executing the transfusion tracking/support software 114 causes the tablet 102 to determine that an adjunct therapeutic product is to be administered to the patient based on a history of blood products administered to the patient. For example, calcium may need to be administered to the patient after every ‘n’ bags of blood products. Accordingly, the tablet 102 may be configured to provide an indication to the user of the adjunct therapeutic product to be administered based on a history of blood products administered to the patient. The recommendation of the adjunct therapeutic product may be in addition to the recommendation of the next blood product to be administered, to maintain the ratio of blood products administered within an acceptable range of the target ratio for the patient.

In some examples, the tablet 102 may be configured to receive physiologic data for the patient that includes a level of the adjunct therapeutic product, such as from an ancillary sensor (e.g., a current ionized calcium value from a bedside diagnostic device) or lab report (e.g., a potassium value from the EMR for that patient). The tablet 102 may thus provide an indication of an amount of the adjunct therapeutic product to be administered based on a treatment protocol and the physiologic data (e.g., the patient’s current level).

In a more specific example, the adjunct therapeutic product is TXA, which is generally not recommended to be administered after a certain amount of time has passed since the patient injury that resulted in the MTP being ordered. Accordingly, executing the transfusion tracking/support software 114 may cause the tablet 102 to receive a user input that indicates a time (or time range) at which the patient injury occurred, which is a first time. The tablet 102 is configured to provide an indication of TXA to be administered to the patient at a second time, such as when the user input that indicates the first time is received by the tablet 102. The indication of TXA to be administered to the patient is based on the duration of a time period between the first time and the second time.

For example, empiric TXA (i.e. TXA administration in the absence of laboratory evidence for fibrinolysis-based blood clot degradation) is recommended to be provided until three hours have elapsed since the patient injury. In a first scenario, the tablet 102 receives a user input that the patient injury occurred less than one hour ago (i.e., the duration is less than one hour), and provides an indication to administer TXA until two hours have passed, which satisfies the rule that TXA not be initiated past three hours from the patient injury. In a second scenario, the tablet 102 receives a user input that the patient injury occurred between one and two hours ago (i.e., the duration is in a range of one to two hours), and provides an indication to administer TXA until one hour has passed, which satisfies the rule that TXA not be provided past three hours from the patient injury. In a third scenario, the tablet 102 receives a user input that the patient injury occurred more than two hours ago (i.e., the duration is more than two hours), and provides an indication to not to administer TXA, which satisfies the rule that empiric TXA not be initiated past three hours from the patient injury.

FIG. 2 is a schematic block diagram of a system 200 for blood transfusion tracking and decision support in accordance with various examples. The system 200 includes a server 202 that is configured to be coupled to one or more computing devices 102a-102n, such as a handheld computing device, including the tablet described above. As above, these computing devices 102a-102n are referred to as tablets for ease of reference. Each of the tablets 102a-102n may be generally configured as described above, including some or all of the functionality provided by the transfusion tracking/support software 114. The tablets 102a-102n are communicably coupled to the server 202 by way of their respective network interfaces 110, such as over one or more networks, which can be wired, wireless, or combinations thereof.

The system 200 also includes a blood bank 204, which is a schematic representation of one or more computing devices and/or one or more blood product management devices, and is configured to be coupled to the server 202. For example, the blood bank 204 includes devices to manage various blood products, such as refrigeration devices, coolers, warming devices (e.g., water baths), and the like. The blood bank 204 also includes computing devices that are configured to send and receive data communications, including control signals, from the server 202. The blood bank 204 may also include blood supply inventory purchasing systems and, as described above, the tablet 102 may be configured to provide a control signal to the blood bank 204 that causes an order to be placed with a supplier of the blood bank 204 to provide an additional blood product.

The system 200 further includes an EMR system 206, which is a schematic representation of one or more computing devices, and is configured to be coupled to the server 202. Accordingly, the server 202 may facilitate various transfusion data being provided to the EMR system 206 by the tablets 102. The server 202 may also facilitate the tablets 102 receiving various data being received from the EMR system 206.

In some examples, the tablet 102 and/or server 202 can be configured to interact with the EMR system 206 (which may also represent or refer to an LIS in some examples), which is configured to store and provide EMR data for various patients. In some examples, the EMR system (or LIS) 206 can be embodied on the server 202 itself. The EMR data may indicate certain laboratory levels for the patient that influence the provision of blood products and/or adjunct therapeutic products to that patient. For example, EMR data may include a blood serum calcium level for the patient and/or physiologic data that indicates levels of other adjunct therapeutic products. Accordingly, the EMR data can be compared to the current ratio of blood products (and adjunct therapeutic products) and/or the history of blood products and adjunct therapeutic products administered to the patient, and an updated indication of the adjunct therapeutic product to be administered to the patient is provided responsive to that comparison. For example, some patients may require calcium to be provided more or less frequently than after every n bags of blood products, and this is reflected in that patient’s EMR data (e.g., by the comparison between EMR data and the current ratio and/or history of blood products and adjunct therapeutic products administered).

In the example of FIG. 1, described above, the tablet 102 generally implements the functionality of the transfusion tracking/support software 114 in a standalone fashion. However, in other examples, the tablet 102 may act as a “front end”, while certain of the processing tasks of the transfusion tracking/support software 114 are carried out by the server 202. For example, the transfusion tracking/support software 114 functionality may generally be implemented by the server and the tablet 102 functions to acquire identifier data (e.g., of the blood product 116, a patient, or other useful identifiers in the clinical setting such as location identifiers) and provide the identifier data to the server 202. In this example, the server 202 determines the blood product(s) to be administered to the patient based on the target ratio of blood products for the particular patient, and a current ratio of blood products already administered to the patient. The server 202 may also update the current ratio of blood products based on an identifier of a blood product 116 received from the tablet 102. The server 202 provides the determined next blood product(s) to the tablet 102, which in turn is configured to provide an indication of the determined next blood product(s) to a user. Optionally, the server 202 may update the EMR system 206 in a corresponding fashion, so that the EMR system 206 can maintain contemporaneous records of various transfusion actions being implemented.

As described above, a control signal may be provided to the equipment at the hospital and/or the blood bank 204 responsive to determining the next blood product, or responsive to a cancellation (e.g., a cancellation order) of a protocol associated with the blood transfusion to the patient, such as the MTP that was ordered for the patient. The tablet 102 may be configured to provide the control signal to the hospital and/or blood bank 204 via the server 202, or the server 202 may be configured to provide the control signal based on data received from the tablet 102.

For example, the control signal may cause an infusion device at the hospital (e.g., an infusion pump, not shown for simplicity) to provide the determined next blood product to the patient in an automated fashion. In this example, the control signal may indicate a type of the determined next blood product, a volume of the determined next blood product, a rate of delivery of the next product, or other values usable by the infusion device to automatically administer the next blood product to the patient.

In another example, the control signal may be provided to the blood bank 204 to cause the blood bank 204 to prepare additional blood product(s) that may be administered to the patient, such as after the determined next blood product. For example, the tablet 102 or the server 202 may determine that, based on the determined next blood product, additional blood product(s) will be needed from the blood bank 204, and thus generates the control signal to preemptively, or more proactively request those additional blood product(s). The control signal may be configured to cause equipment at the blood bank 204 to begin warming the additional blood product(s) or otherwise preparing the blood product(s) for transport and administration to the patient.

In yet another example, the control signal may be provided to the blood bank 204 to cause the blood bank 204 to stop preparing additional blood products according to the MTP order for the patient, such as responsive to a cancellation of the MTP that is indicated (e.g., by a user input) to the tablet 102, which may be received and forwarded by the server 202. In some cases, the cancellation of an MTP may be relayed to the blood bank 204 in a delayed or otherwise ineffective manner, which can result in the blood bank 204 preparing blood products for a now-cancelled order, thus leading to spoilage of those blood products. However, as a result of providing a specific cancellation control signal by the tablet 102 and/or server 202, the cancellation of the MTP is more effectively communicated to the blood bank 204 (including in an automated fashion in some examples), which may reduce or eliminate blood product spoilage by more effectively and directly communicating the cancellation of the MTP and causing blood bank 204 personnel and/or equipment to modify their behavior accordingly.

In still another example, the control signal may be provided to the blood bank 204 to cause the blood bank 204 (or a blood supply inventory purchasing system thereof) to place an order with a supplier of the blood bank 204 to provide an additional blood product.

In some examples, the system 200 (e.g., the tablet 102 thereof) is configured to apply a time stamp to the acquired blood product data, so that the time at which the blood product transfusion is started is also captured.

In the distributed example of FIG. 2, the server 202 is configured to count and save the total number of blood products administered to the patient (e.g., to update the total number of blood products based on the scanned data associated with the blood product being administered to the patient) based on data received from the tablet 102. The server 202 may also be configured to count and save the ratio of blood products given (e.g., to update the current ratio based on the scanned data associated with the blood product being administered to the patient) based on data received from the tablet 102. As described above, various data such as total blood products or the current ratio of blood products administered to the patient may be displayed to a user, such as on a GUI of the tablet 102 in the system 200.

As described above, the tablet 102 in the system 200 is configured to provide visual highlights and/or textual prompts of a determined next blood product in order to maintain a balanced resuscitation (e.g., according to the MTP). The tablet 102 in the system 200 can also provide textual prompts (e.g., clinical decision support recommendations) for adjunct therapeutic products to be given empirically (e.g., one unit of calcium chloride after every four units of blood products have been given), and can save and record adjunct therapeutic products given, along with their corresponding timestamps.

The tablet 102 may be configured to receive authentication information of a user, such as a user name/password combination, a unique identifier of a user, or other information related to a user of the tablet 102. Accordingly, the tablet 102 in either of FIGS. 1 or 2 is configured to facilitate dual verification of blood products prior to administering to the patient (e.g., transfusion). As above, the tablet 102 may collect this information using a barcode scanner, an on-screen keyboard, the imaging device thereof, or an RF transceiver. In some examples, the tablet 102 is configured to acquire the user identification information for each blood product administered to the patient, or to acquire user identification information and to store the information, which may be attributed to multiple products of blood, until the user of the tablet 102 disassociates their user identification information from further dual verification events.

Various settings of the system 200 may be user-configurable, which allows the system 200 to be used in different contexts having different clinical preferences. For example, the target ratio of blood products can be user-configurable. In other examples, the target ratio of blood products may be varied based on other factors, such as a type of patient being cared for (e.g., a maternity patient, versus a vascular surgery patient, versus a trauma patient, and the like), a patient age and/or weight, and other patient factors. In other examples, various other settings that impact the administration of blood products and/or adjunct therapeutic products are also user-configurable.

In an example, the server 202 of the system 200 is a web server configured to execute software that retains records or logs of data related to blood transfusions being administered in conjunction with the tablet 102 (e.g., computing device) of the system 200.

The web server 202 can be configured to authenticate users (e.g., users of the tablets 102a-102n) to provide additional security and privacy for data stored on the web server 202 and/or the tablet 102.

In some examples, the tablet 102 (or transfusion tracking/support software 114 executed thereon) stores massive transfusion scores in a checklist format. The massive transfusion scores enable a recommendation to the clinical provider to initiate a massive transfusion order based on a certain number of checkboxes being checked, or a certain combination of clinical values being entered.

In some examples, the tablet 102 (or transfusion tracking/support software 114 executed thereon) provides a timer to track events (e.g., administration of blood products and/or adjunct therapeutic products) by clock time as well as by relative time, such as since the software 114 began executing.

In some examples, the tablet 102 (or transfusion tracking/support software 114 executed thereon) includes buttons that allow the user to indicate when the massive transfusion order was started and when it was stopped, and when hemostasis was occurred (or when hemostasis was later discovered to not have occurred). These events can also time stamped.

In some examples, the web server 202 is configured to provide real-time (e.g., within a few seconds) status alerts to blood bank 204 technicians. Accordingly, the web server 202 is configured to provide visual and/or audio alerts to a computer or tablet near the blood bank 204 technicians, which may signify various events, such as when a blood product is given, when a set or cooler of blood products has been given, when hemostasis is achieved, when the clinical user has indicated that the physician has cancelled the massive transfusion order as described above.

The web server 202 may be configured to generate data files in various formats (e.g., PDF and CSV files), which allow users to download the data in easily storable files for after-action reviews, EMR recordkeeping, performance improvement, and research.

As described, in some examples, the blood product data is scanned with either a handheld barcode scanner, by RFID tag (if the data is stored in an RFID tag), or by optical decoding by the tablet 102 imaging device 112. The web server 202 or tablet 102 may communicate with the EMR system 206 to automatically send transfusion data to the EMR system 206, which may include MRNs, blood product label data, time stamp data, adjunct data, and dual verification clinician identifiers. The web server 202 or tablet 102 may communicate with the EMR system or Laboratory Information System (LIS) 206 to receive laboratory data, such as blood chemistries, blood test data, VHA and coagulopathy data, and to provide recommendations for blood products or adjuncts based on the EMR or LIS data.

In some cases, the functionality described herein is provided by (e.g., integrated with) the EMR system 206 itself. Accordingly, in these cases, a separate tablet 102 and web server 202 may not be needed and, instead, the server(s) and/or computing device(s) (e.g., tablet(s)) of the EMR system 206 are configured to perform the functionality described herein.

The tablet 102 may be wirelessly connected to a vital signs monitor, which may automate some massive transfusion scoring (e.g., for deciding when to initiate a massive transfusion protocol). Additionally, the tablet 102 may include massive transfusion score checklists. The 102 tablet may also include a machine-learning algorithm, which provides a probabilistic estimate for whether a patient will need at least ‘x’ units of a blood product (e.g., red blood cells) within a ‘y’ minute period (e.g., at least three units of red blood cells in a 60-minute period).

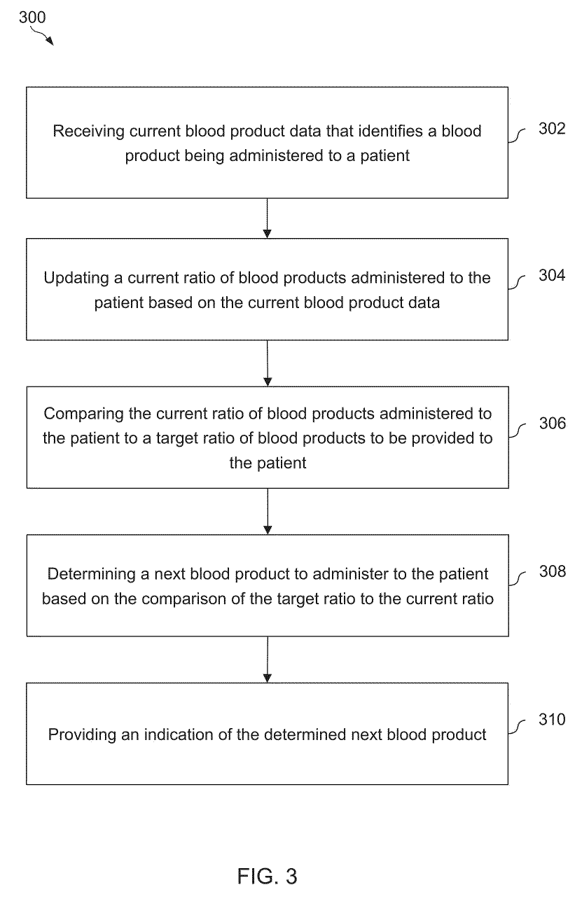

FIG. 3 is a flow chart of a method 300 for providing blood transfusion tracking and decision support in accordance with various examples. The method 300 begins in block 302 with receiving current blood product data that identifies a blood product being administered to the patient. As described above, current blood product data identifies a blood product being administered to the patient. For example, a barcode of a bag containing the blood product may be scanned (e.g., by the tablet 102 or by a scanning device coupled thereto), which provides data that identifies the blood product to the tablet 102. In another example, a radio-frequency identification (RFID) tag associated with the bag containing the blood product may be scanned (e.g., by the tablet 102 or by a RFID scanner coupled thereto), which provides data that identifies the blood product to the tablet 102. The blood product may be considered as being administered to the patient once it has been scanned, even though the blood product is not yet physically administered to the patient.

The method 300 continues in block 304 with updating a current ratio of blood products administered to the patient based on the current blood product data. Before any blood products have been administered to the patient, the current ratio may simply be updated (e.g., from 0 or a default value) to include the current blood product data. Subsequently, the current ratio is updated to add the current blood product data to the previously-provided ratio of blood products. The updated current ratio may be stored in a memory 106 of the tablet 102, provided to the server 202 for storage thereon, or both.

The method 300 continues further in block 306 with comparing the current ratio of blood products administered to the patient to a target ratio of blood products to be provided to the patient. The method 300 then continues in block 308 with determining a next blood product to administer to the patient based on the comparison of the target ratio to the current ratio. Finally, the method 300 continues in block 310 with providing an indication of the determined next blood product. For example, if the current ratio indicates that additional plasma should be administered to bring the balance of blood products closer to the target ratio, the indication is for plasma. Similarly, if the current ratio indicates that additional platelets should be administered to bring the balance of blood products closer to the target ratio, the indication is for platelets. In some examples, if the current blood product data identifies an unexpected blood product (e.g., a prior recommendation was for a first blood product, and the blood product being administered, or that has just been scanned, is a second blood product), an alert may be provided (e.g., on the tablet GUI) to indicate to the user that the blood product is not the appropriate one to be administered at this point in the patient’s treatment.

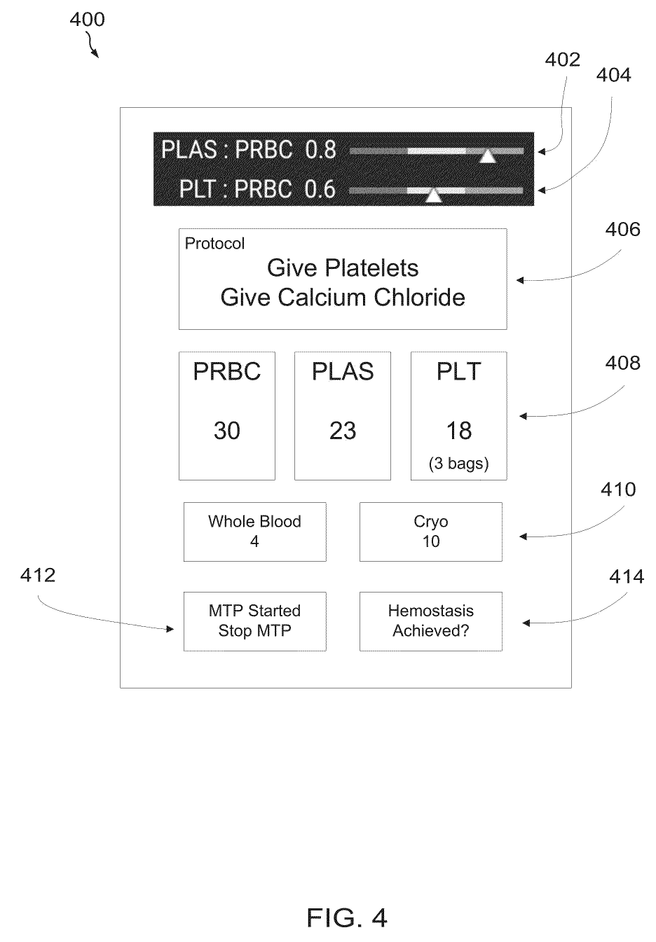

FIG. 4 is an example screenshot 400 of a computing device (e.g., tablet 102) that is configured to provide blood transfusion tracking and decision support in accordance with various examples, and as described above. The screenshot 400 includes example fields that demonstrate certain parts of the functionality as described above. For example, the screenshot 400 includes a first graphic 402 that is an indication of a ratio of plasma (PLAS) to packed red blood cells (PRBC). In this example, the first graphic 402 indicates that the ratio of PLAS:PRBC is in an acceptable range.

The screenshot 400 also includes a second graphic 404 that is an indication of a ratio of platelets (PLT) to PRBC. In this example, the second graphic 404 indicates that the ratio of PLT:PRBC is lower than expected, which may indicate to a clinician that additional platelets should be provided to the patient.

Accordingly, the screenshot 400 also includes a third graphic 406 that provides an indication to give additional platelets to the patient. As described above, it may also be determined that certain adjunct therapeutic products should be given to the patient, and thus the third graphic 406 also provides an indication to give additional calcium chloride to the patient.

The screenshot 400 also includes a fourth graphic 408 that indicates a current ratio of blood products administered to the patient. In the example of FIG. 4, the current ratio indicated by the fourth graphic 408 includes PRBC, PLAS, and PLT values, which may correspond to a number of units of each that have been provided to the patient. Also, in the example of FIG. 4, the fourth graphic 408 indicates a number of bags that are associated with the PLT count (e.g., where multiple apheresis platelet units are stored within a single bag).

The screenshot 400 also includes a fifth graphic 410 that includes a whole blood count of 4 and a cryoprecipitate count of 10. The screenshot 400 further includes a sixth graphic 412, which is a Stop MTP button 412 that includes an indication of whether to stop an MTP (e.g., responsive to the Stop MTP button being pressed). Responsive to the Stop MTP button 412 being pressed, the computing device 102 is configured to provide a control signal to the hospital and/or blood bank 204 (e.g., via the server 202, or is configured to cause the server 202 to provide the control signal based on data received from the tablet 102.

In this example, the control signal is provided to the blood bank 204 to cause the blood bank 204 to stop preparing additional blood products, because the user input of pressing the Stop MTP button 412 indicates cancellation of the MTP. As a result of providing a specific cancellation control signal by the tablet 102 and/or server 202, the cancellation of the MTP is more effectively communicated to the blood bank 204 (including in an automated fashion in some examples), which may reduce or eliminate blood product spoilage by more effectively and directly communicating the cancellation of the MTP and causing blood bank 204 personnel and/or equipment to modify their behavior accordingly.

The screenshot 400 also includes a seventh graphic 414, which is an indication of whether hemostasis has been achieved. For example, the seventh graphic 414 may be highlighted or otherwise presented in a manner that affirms that hemostasis has been achieved, and may be greyed out or otherwise presented in a manner that suggests hemostasis has not yet been achieved.

The term “couple” is used throughout the specification. The term may cover connections, communications, or signal paths that enable a functional relationship consistent with this description. For example, if device A generates a signal to control device B to perform an action, in a first example device A is coupled to device B, or in a second example device A is coupled to device B through intervening component C if intervening component C does not substantially alter the functional relationship between device A and device B such that device B is controlled by device A via the control signal generated by device A.

A device that is “configured to” perform a task or function may be configured (e.g., programmed and/or hardwired) at a time of manufacturing by a manufacturer to perform the function and/or may be configurable (or re-configurable) by a user after manufacturing to perform the function and/or other additional or alternative functions. The configuring may be through firmware and/or software programming of the device, through a construction and/or layout of hardware components and interconnections of the device, or a combination thereof. A circuit or device that is described herein as including certain components may instead be adapted to be coupled to those components to form the described circuitry or device. Unless otherwise stated, “about,” “approximately,” or “substantially” preceding a value means +/- 10 percent of the stated value. Modifications are possible in the described examples, and other examples are possible within the scope of the claims.

What is claimed is:

1. A method for tracking and supporting a blood transfusion to a patient, the method comprising:
receiving, by a computing device, current blood product data that identifies a blood product being administered to the patient;
updating a current ratio of blood products administered to the patient based on the current blood product data;
comparing the current ratio of blood products administered to the patient to a target ratio of blood products to be provided to the patient;
determining a next blood product to administer to the patient based on the comparison of the target ratio to the current ratio; and
providing, by the computing device, an indication of the determined next blood product.

2. The method of claim 1, comprising providing an indication of the current ratio.

3. The method of claim 1, further comprising providing a control signal to cause an infusion device to provide the determined next blood product to the patient, wherein the control signal indicates a type of the determined next blood product, a volume of the next blood product, a rate of delivery of the next blood product, or combinations thereof.

4. The method of claim 1, further comprising providing a control signal to a blood bank to cause the blood bank to prepare an additional blood product responsive to the determined next blood product.

5. The method of claim 4, wherein the control signal is a first control signal, the method further comprising providing a second control signal to the blood bank to cause the blood bank to stop preparing additional blood products responsive to a cancellation of a transfusion protocol.

6. The method of claim 1, wherein the target ratio is specified by a user input as a setting for the transfusion, or wherein the target ratio is determined based on one or more physiological parameters of the patient.

7. The method of claim 1, further comprising:
receiving patient identification data associated with the patient; and
providing an indication of a total number of blood products administered to the patient responsive to the patient identification data.

8. The method of claim 1, further comprising:
determining an adjunct therapeutic product to be administered to the patient based on a history of blood products administered to the patient; and
providing an indication of the adjunct therapeutic product to be administered to the patient.

9. The method of claim 8, further comprising:
receiving physiologic data for the patient that includes a level of the adjunct therapeutic product for the patient; and
providing an indication of an amount of the adjunct therapeutic product to be administered based on a treatment protocol and the physiologic data.

10. The method of claim 8, wherein the adjunct therapeutic product is tranexamic acid (TXA), the method further comprising receiving an indication of a first time of an injury of the patient, wherein the blood transfusion is in response to the injury, wherein the indication of TXA to be administered to the patient is provided at a second time, and wherein the indication of TXA to be administered to the patient is based on a duration of a time period between the first time and the second time.

11. The method of claim 1, further comprising:
comparing the current blood product data to previously received blood product data; and
updating the current ratio of blood products administered to the patient responsive to determining that the current blood product data is not duplicate data; or
not updating the current ratio of blood products administered to the patient responsive to determining that the current blood product data is duplicate data.

12. A non-transitory, computer-readable medium containing instructions that, when executed by a processor of a computing device, cause the computing device to be configured to:
receive current blood product data that identifies a blood product being administered to a patient;
update a current ratio of blood products administered to the patient based on the current blood product data;
compare the current ratio of blood products administered to the patient to a target ratio of blood products to be provided to the patient;
determine a next blood product to administer to the patient based on the comparison of the target ratio to the current ratio; and
provide an indication of the determined next blood product.

13. The non-transitory, computer-readable medium of claim 12, wherein the instructions, when executed by the processor, cause the computing device to be configured to provide a control signal to cause an infusion device to provide the determined next blood product to the patient, wherein the control signal indicates a type of the determined next blood product, a volume of the next blood product, a rate of delivery of the next blood product, or combinations thereof.

14. The non-transitory, computer-readable medium of claim 12, wherein the instructions, when executed by the processor, cause the computing device to be configured to:
provide a first control signal to a blood bank to cause the blood bank to prepare an additional blood product responsive to the determined next blood product; and
provide a second control signal to the blood bank to cause the blood bank to stop preparing additional blood products responsive to a cancellation of a transfusion protocol.

15. The non-transitory, computer-readable medium of claim 12, wherein the instructions, when executed by the processor, cause the computing device to be configured to:
receive patient identification data associated with the patient; and
provide an indication of a total number of blood products administered to the patient responsive to the patient identification data.

16. The non-transitory, computer-readable medium of claim 12, wherein the instructions, when executed by the processor, cause the computing device to be configured to:
compare the current blood product data to previously received blood product data; and
update the current ratio of blood products administered to the patient responsive to determining that the current blood product data is not duplicate data; or
not update the current ratio of blood products administered to the patient responsive to determining that the current blood product data is duplicate data.

17. A device, comprising:
a processor;
a display unit coupled to the processor; and
a memory coupled to the processor and configured to store instructions that, when executed by the processor, cause the device to be configured to:
receive current blood product data that identifies a blood product being administered to a patient;
update a current ratio of blood products administered to the patient based on the current blood product data;
compare the current ratio of blood products administered to the patient to a target ratio of blood products to be provided to the patient;
determine a next blood product to administer to the patient based on the comparison of the target ratio to the current ratio; and
provide an indication of the determined next blood product.

18. The device of claim 17, wherein the instructions, when executed by the processor, cause the device to be configured to provide a control signal to cause an infusion device to provide the determined next blood product to the patient, wherein the control signal indicates a type of the determined next blood product, a volume of the next blood product, a rate of delivery of the next blood product, or combinations thereof.

19. The device of claim 17, wherein the instructions, when executed by the processor, cause the device to be configured to:
provide a first control signal to a blood bank to cause the blood bank to prepare an additional blood product responsive to the determined next blood product; and
provide a second control signal to the blood bank to cause the blood bank to stop preparing additional blood products responsive to a cancellation of a transfusion protocol.

20. The device of claim 17, wherein the instructions, when executed by the processor, cause the device to be configured to:
compare the current blood product data to previously received blood product data; and
update the current ratio of blood products administered to the patient responsive to determining that the current blood product data is not duplicate data; or
not update the current ratio of blood products administered to the patient responsive to determining that the current blood product data is duplicate data.
